(19) United States
(12) Patent Application Publication
(10) Pub. No.: US 2026/0325434 A1
(43) Pub. Date: Oct. 22, 2026

(54) SKIN CARE COMPOSITION

(71) Applicant: GH Bluebird Holdings INC, Boca Raton, FL (US)

(72) Inventor: Stacey BERGER, Boca Raton, FL (US)

(21) Appl. No.: 19/541,978

(22) Filed: Feb. 17, 2026

Related U.S. Application Data
(60) Provisional application No. 63/759,744, filed on Feb. 18, 2025.

(51) Int. Cl. A61K 8/9728; A61K 8/34; A61K 8/64; A61K 8/9789; A61K 8/9794; A61Q 19/00; A61Q 19/02; A61Q 19/08

(52) U.S. Cl. CPC A61K 8/9728; A61K 8/345; A61K 8/64; A61K 8/9789; A61K 8/9794; A61Q 19/00; A61Q 19/007; A61Q 19/02; A61Q 19/08; A61K 2800/522; A61K 2800/5922

(57) ABSTRACT

The present disclosure relates to a synergistic skin care complex combining phytoactive botanical extracts, bioactive peptides, and adaptogenic plant-derived extracts.

### CROSS REFERENCE TO RELATED APPLICATIONS

The present application claims benefit to U.S. Provisional Patent Application No. 63/759,744, filed Feb. 18, 2025, the disclosure of which is incorporated by reference herein in its entirety.

### FIELD OF THE INVENTION

The present disclosure relates to a synergistic skin care complex combining phytoactive botanical extracts, bioactive peptides, and adaptogenic plant-derived extracts.

### SUMMARY OF THE INVENTION

In some embodiments, the present disclosure provides a composition comprising a botanical extract blend comprising a botanical extract blend comprising Punica granatum fruit extract at about 0.5% to about 5% by weight of the blend; Papaver rhoeas petal extract at about 0.1% to about 3% by weight of the blend; Ziziphus jujuba fruit extract at about 0.1% to about 3% by weight of the blend; Trifolium pratense (clover) leaf extract at about 0.1% to about 3% by weight of the blend; Cimicifuga racemosa root extract at about 0.1% to about 3% by weight of the blend; Lithospermum erythrorhizon root extract at about 0.1% to about 3% by weight of the blend; and Dioscorea japonica root extract at about 0.1% to about 3% by weight of the blend; a bioactive peptide complex comprising acetyl tetrapeptide 11 at about 0.0525% to about 0.1% by weight of the complex and acetyl tetrapeptide 9 at about 0.35% to about 1% by weight of the complex; an adaptogenic plant-derived extract comprising propanediol and Inonotus obliquus (mushroom) extract at about 0.5% to about 5% by weight of the extract; and the remainder of the composition comprising one or more of a solvent, a preservative and an excipient.

In some embodiments, the botanical extract blend comprises Punica granatumfruit extract at about 1.5% to about 3% by weight of the blend. In some embodiments, the botanical extract blend comprises Papaver rhoeas petal extract at about 1% to about 2% by weight of the blend. In some embodiments, the botanical extract blend comprises Ziziphus jujuba fruit extract at about 1% to about 2% by weight of the blend. In some embodiments, the botanical extract blend comprises Trifolium pratense (clover) leaf extract at about 1% to about 2% by weight of the blend. In some embodiments, the botanical extract blend comprises Cimicifuga racemosa root extract at about 1% to about 2% by weight of the blend. In some embodiments, the botanical extract blend comprises Lithospermum erythrorhizonroot extract at about 1% to about 2% by weight of the blend. In some embodiments, the botanical extract blend comprises Dioscorea japonica root extract at about 1% to about 2% by weight of the blend.

In some embodiments, the bioactive peptide complex comprises acetyl tetrapeptide 11 at about 0.065% to about 0.085% by weight of the complex. In some embodiments, the bioactive peptide complex comprises acetyl tetrapeptide 9 at about 0.50% to about 0.80% by weight of the complex. In some embodiments, the adaptogenic plant-derived extract comprises propanediol and Inonotus obliquus (mushroom) extract at about 1.5% to about 3.0% by weight of the extract.

### DETAILED DESCRIPTION OF THE INVENTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

In various embodiments, the present disclosure provides a composition comprising botanical extracts, a bioactive peptide complex, and an adaptogenic plant-derived extract. This synergistic cosmetic complex combines phytoactive botanical extracts, bioactive peptides, and adaptogenic plant-derived extracts in optimized ratios to deliver enhanced skin rejuvenation, hydration, and anti-aging benefits. The combination targets multiple mechanisms of skin aging, providing collagen stimulation, antioxidant protection, and enhanced moisture retention.

The botanical extract blend is a phytoactive component designed to provide phytoestrogenic, antioxidant, and anti-inflammatory benefits. The components are included in ranges that optimize skin balance and collagen synthesis. The botanical extract blend enhances the extracellular matrix and fibroblast stimulation, promoting wrinkle reduction and improved skin tone. The peptide complex works synergistically with the botanical extracts to improve skin density and elasticity and promote collagen. The adaptogenic plant-derived extract is rich in polysaccharides and antioxidants, helping to reduce oxidative stress and inflammation. The combination of botanical phytoestrogens, bioactive peptides, and adaptogens enhances skin health through synergistic mechanisms. The components of the composition provide (i) increased collagen synthesis and fibroblast activity from phytoestrogens and peptides working synergistically, (ii) Enhanced moisture retention and skin barrier protection from hydrating botanical extracts and propanediol, and (iii) reduced inflammation and oxidative stress from anti-inflammatory and antioxidant plant actives. The cosmetic composition provides functional benefits such as (i) immediate calming of inflammation through adaptogenic and antioxidant activity, (ii) long-term improvement in elasticity and wrinkle reduction from enhanced collagen production, and (iii) improved hydration and elasticity compared to individual components.

In some embodiments, the present disclosure provides a composition comprising a botanical extract blend. In some embodiments, the botanical extract blend comprises Punica granatum fruit extract. Punica granatum fruit extract is included in the blend for its antioxidant properties and collagen support. In some embodiments, the blend comprises Punica granatum fruit extract at about 0.5% to about 5% by weight. In some embodiments, the blend comprises Punica granatum fruit extract at about 1.0% to about 4.0% by weight. In some embodiments, the blend comprises Punica granatum fruit extract at about 1.5% to about 3.0% by weight. In some embodiments, the blend comprises Punica granatum fruit extract at about 2.0% to about 2.5% by weight.

In some embodiments, the botanical extract blend further comprises Papaver rhoeas petal extract. Papaver rhoeas petal extract is included in the blend for its anti-inflammatory and antioxidant properties. In some embodiments, the blend comprises Papaver rhoeas petal extract at about 0.1% to about 3% by weight. In some embodiments, the blend comprises Papaver rhoeas petal extract at about 0.5% to about 2.5% by weight. In some embodiments, the blend comprises Papaver rhoeas petal extract at about 1% to about 2% by weight. In some embodiments, the blend comprises Papaver rhoeas petal extract at about 1.3% to about 1.7% by weight.

In some embodiments, the botanical extract blend further comprises Ziziphus jujuba fruit extract. Ziziphus jujuba fruit extract is included in the blend for its wound-healing and soothing effects. In some embodiments, the blend comprises Ziziphus jujuba fruit extract at about 0.1% to about 3% by weight. In some embodiments, the blend comprises Ziziphus jujuba fruit extract at about 0.5% to about 2.5% by weight. In some embodiments, the blend comprises Ziziphus jujuba fruit extract at about 1% to about 2% by weight. In some embodiments, the blend comprises Ziziphus jujuba fruit extract at about 1.3% to about 1.7% by weight.

In some embodiments, the botanical extract blend further comprises Trifolium pratense (clover) leaf extract. Trifolium pratense (clover) leaf extract is included in the blend for its phytoestrogens that promote skin elasticity. In some embodiments, the blend comprises Trifolium pratense (clover) leaf extract at about 0.1% to about 3% by weight. In some embodiments, the blend comprises Trifolium pratense (clover) leaf extract at about 0.5% to about 2.5% by weight. In some embodiments, the blend comprises Trifolium pratense(clover) leaf extract at about 1% to about 2% by weight. In some embodiments, the blend comprises Trifolium pratense (clover) leaf extract at about 1.3% to about 1.7% by weight.

In some embodiments, the botanical extract blend further comprises Cimicifuga racemosa root extract. Cimicifuga racemosa root extract is included in the blend for its phytoestrogens that promote collagen synthesis. In some embodiments, the blend comprises Cimicifuga racemosa root extract at about 0.1% to about 3% by weight. In some embodiments, the blend comprises Cimicifuga racemosa root extract at about 0.5% to about 2.5% by weight. In some embodiments, the blend comprises Cimicifuga racemosa root extract at about 1% to about 2% by weight. In some embodiments, the blend comprises Cimicifuga racemosa root extract at about 1.3% to about 1.7% by weight.

In some embodiments, the botanical extract blend further comprises Lithospermum erythrorhizon root extract. Lithospermum erythrorhizon root extract is included in the blend for its anti-inflammatory and antibacterial properties. In some embodiments, the blend comprises Lithospermum erythrorhizon root extract at about 0.1% to about 3% by weight. In some embodiments, the blend comprises Lithospermum erythrorhizon root extract at about 0.5% to about 2.5% by weight. In some embodiments, the blend comprises Lithospermum erythrorhizon root extract at about 1% to about 2% by weight. In some embodiments, the blend comprises Lithospermum erythrorhizon root extract at about 1.3% to about 1.7% by weight.

In some embodiments, the botanical extract blend further comprises Dioscorea japonica root extract. Dioscorea japonica root extract is included in the blend for its function in skin hydration and hormonal balance. In some embodiments, the blend comprises Dioscorea japonica root extract at about 0.1% to about 3% by weight. In some embodiments, the blend comprises Dioscorea japonica root extract at about 0.5% to about 2.5% by weight. In some embodiments, the blend comprises Dioscorea japonica root extract at about 1% to about 2% by weight. In some embodiments, the blend comprises Dioscorea japonica root extract at about 1.3% to about 1.7% by weight.

In some embodiments, the present disclosure provides a composition further comprising a bioactive peptide complex. In some embodiments, the bioactive peptide complex comprises acetyl tetrapeptide 11. Acetyl tetrapeptide 11 is included in the complex for its function in stimulating collagen types I and XVII. In some embodiments, the complex comprises acetyl tetrapeptide 11 at about 0.0525% to about 0.1% by weight. In some embodiments, the complex comprises acetyl tetrapeptide 11 at about 0.060% to about 0.090% by weight. In some embodiments, the complex comprises acetyl tetrapeptide 11 at about 0.065% to about 0.085% by weight. In some embodiments, the complex comprises acetyl tetrapeptide 11 at about 0.070% to about 0.080% by weight.

In some embodiments, the bioactive peptide complex further comprises acetyl tetrapeptide 9. Acetyl tetrapeptide 9 is included in the complex for its function in enhancing fibroblast activity and structural integrity. In some embodiments, the complex comprises acetyl tetrapeptide 9 at about 0.35% to about 1% by weight. In some embodiments, the complex comprises acetyl tetrapeptide 9 at about 0.40% to about 0.090% by weight. In some embodiments, the complex comprises acetyl tetrapeptide 9at about 0.50% to about 0.80% by weight. In some embodiments, the complex comprises acetyl tetrapeptide 9 at about 0.60% to about 0.70% by weight.

In some embodiments, the present disclosure provides a composition further comprising an adaptogenic plant-derived extract. In some embodiments, the adaptogenic plant-derived extract comprises propanediol and Inonotus obliquus (mushroom) extract. Propanediol and Inonotus obliquus (mushroom) extract is included in the extract for its anti-inflammatory and antioxidant properties. In some embodiments, the plant-derived extract comprises propanediol and Inonotus obliquus (mushroom) extract at about 0.5% to about 5% by weight. In some embodiments, the plant-derived extract comprises propanediol and Inonotus obliquus (mushroom) extract at about 1% to about 4% by weight. In some embodiments, the plant-derived extract comprises propanediol and Inonotus obliquus (mushroom) extract at about 1.5% to about 3% by weight. In some embodiments, the plant-derived extract comprises propanediol and Inonotus obliquus (mushroom) extract at about 2% to about 2.5% by weight.

Suitably, the remainder of the compositions described herein further comprise one or more of a solvent, a preservative and an excipient.

In some embodiments, the components of the composition are each dissolved in a solvent, such as water, oil, or organic solvent (e.g. methanol, ethanol, acetonitrile) and the like. Additional components include various preservatives and other excipients, including buffering agents and the like. The compositions of the present disclosure may be in a wide variety of product forms that include, but are not limited to, solutions, suspensions, lotions, creams, gels, sprays, ointments and serums. In some embodiments, the components of the composition are present in a product at a concentration of about 1% to about 10% by weight.

The present invention is not limited to the specific embodiments described above, but rather encompasses all variations and combinations thereof that are within the scope of the claims.

What is claimed is:

1. A composition comprising:
a. a botanical extract blend comprising:
i. Punica granatum fruit extract at about 0.5% to about 5% by weight of the blend;
ii. Papaver rhoeas petal extract at about 0.1% to about 3% by weight of the blend;
iii. Ziziphus jujuba fruit extract at about 0.1% to about 3% by weight of the blend;
iv. Trifolium pratense (clover) leaf extract at about 0.1% to about 3% by weight of the blend;
v. Cimicifuga racemosa root extract about 0.1% to about 3% by weight of the blend;
vi. Lithospermum erythrorhizon root extract at about 0.1% to about 3% by weight of the blend; and
vii. Dioscorea japonica root extract at about 0.1% to about 3% by weight of the blend;
b. a bioactive peptide complex comprising:
i. acetyl tetrapeptide 11 at about 0.0525% to about 0.1% by weight of the complex; and
ii. acetyl tetrapeptide 9 at about 0.35% to about 1% by weight of the complex;
c. an adaptogenic plant-derived extract comprising propanediol and Inonotus obliquus (mushroom) extract at about 0.5% to about 5% by weight of the extract; and
d. remainder of the composition comprising one or more of a solvent, a preservative and an excipient.

2. The composition of claim 1, wherein the botanical extract blend comprises Punica granatum fruit extract at about 1.5% to about 3% by weight of the blend.

3. The composition of claim 1, wherein the botanical extract blend comprises Papaver rhoeas petal extract at about 1% to about 2% by weight of the blend.

4. The composition of claim 1, wherein the botanical extract blend comprises Ziziphus jujuba fruit extract at about 1% to about 2% by weight of the blend.

5. The composition of claim 1, wherein the botanical extract blend comprises Trifolium pratense (clover) leaf extract at about 1% to about 2% by weight of the blend.

6. The composition of claim 1, wherein the botanical extract blend comprises Cimicifuga racemosa root extract at about 1% to about 2% by weight of the blend.

7. The composition of claim 1, wherein the botanical extract blend comprises Lithospermum erythrorhizon root extract at about 1% to about 2% by weight of the blend.

8. The composition of claim 1, wherein the botanical extract blend comprises Dioscorea japonica root extract at about 1% to about 2% by weight of the blend.

9. The composition of claim 1, wherein the bioactive peptide complex comprises acetyl tetrapeptide 11 at about 0.065% to about 0.085% by weight of the complex.

10. The composition of claim 1, wherein the bioactive peptide complex comprises acetyl tetrapeptide 9 at about 0.50% to about 0.80% by weight of the complex.

11. The composition of claim 1, wherein the adaptogenic plant-derived extract comprises propanediol and Inonotus obliquus (mushroom) extract at about 1.5% to about 3.0% by weight of the extract.
